(19) United States
(12) Patent Application Publication
(10) Pub. No.: US 2017/0079665 A1
(43) Pub. Date: Mar. 23, 2017

(54) Quick-Release Digit Tourniquet and Associated Methods

(71) Applicant: ASep Healthcare Limited, Liverpool (GB)

(72) Inventor: Christian Victor Fellowes, London (GB)

(73) Assignee: ASep Healthcare Limited, Liverpool (GB)

(21) Appl. No.: 14/868,527

(22) Filed: Sep. 29, 2015

(30) Foreign Application Priority Data
Sep. 18, 2015 (GB) 1516581.4

(51) Int. Cl. A61B 17/132

(52) U.S. Cl. CPC A61B 17/1322; A61B 2017/0023

(57) ABSTRACT

A quick-release digit tourniquet having first and second portions of a main body which are engageable for the formation of a loop around a digit of a patient. The first and second portions are interposed by a release element having weakened areas, hereby release perforations, which may be broken or ruptured by the manual application of a force by a user. In particular, the force may be applied to a tab forming part of the release element, simplifying application. Once ruptured, the main body is split into multiple parts and the quick-release digit tourniquet is released from the patient.

### FIELD OF THE TECHNOLOGY

The present invention relates to a quick-release tourniquet, more particularly a quick-release digit tourniquet for simplified removal from a digit, a method of use of such a tourniquet, and a method of automatically rupturing such a tourniquet when exposed to more than a predetermined tension.

### BACKGROUND TO THE INVENTION

This application claims priority to UK patent application number 1516581.4 filed on Sep. 18, 2015, the disclosure of which is incorporated herein by reference.

Tourniquets are devices used to compress or constrict blood vessels of a patient, generally to provide haemostasis, typically being to arrest blood circulation. A simple tourniquet consists of a strip of material which can be tied around a limb or extremity, above the site where blood flow restriction is required. Such a device is beneficial for a bloodless operating field.

However, the use of tourniquets has not always been popular and is commonly only used as a last resort. Prolonged use of a tourniquet, or the application of a tourniquet too tightly, can result in irreversible damage to blood vessels and/or nerves. Following this, tissue death, necrosis, or altered sensation may occur below the site of the tourniquet.

Another issue with the use of tourniquets is that initiating their release once they are no longer required, which is critical for the above reasons. For instance, untying a simple tourniquet may be difficult or time-consuming, due to the tightness of engagement. In non-blood loss-related treatments, such as cannulation or surgery, it is even more critical that the blood flow is not completely cut-off during insertion of the cannula, and that blood flow can be swiftly restored.

On digit or digital tourniquets in particular, which are applied to digits such as fingers, thumbs and toes, or to the penis, excessive pressure or prolonged time of application on what are relatively fragile body parts may lead to neurovascular damage. Therefore, the above issues may be especially prevalent in relation to digital injuries.

There have been reports of tourniquets being left in situ, stopping blood flow to the digit and ultimately causing the death and necrosis to the digit.

It is an object of the present invention to prevent or limit the above issues, by providing an improved tourniquet and methods of use.

### SUMMARY OF THE INVENTION

According to a first aspect of the present invention there is provided a quick-release digit tourniquet for simplified removal from a digit, the digit tourniquet comprising: an elongate main body of an inextensible or substantially inextensible material having interengagable first and second portions; an engagement region, located on the first portion, through which the second portion may pass; and a release element interposed between the first and second portions for weakening the main body such that a transverse of the main body is formed under application of a predetermined external force.

The release element of the present invention allows a user to swiftly and safely rupture or break the main body of the quick-release digit tourniquet into at least two parts. Thus, the quick-release digit tourniquet may be instantly removed without any substantial motion of the tourniquet relative to the digit, pre-release.

Preferably, the quick-release digit tourniquet may further comprise an engagement region, located on the first portion, through which the second portion may pass. It may be advantageous to further provide a fastener on the main body for securing the second portion once it has passed through the engagement region, resulting in a fastened condition.

The provision of the engagement region and fastener may enhance the engagement of the first and second portions in order to secure the quick-release digit tourniquet around a digit. This can make application of the tourniquet quicker and easier, without having to resort to tying or other sub-standard application techniques.

In a beneficial arrangement, the fastener may include an adhesive portion, having an adhesive thereon, disposed on a face of the main body. Thus, the main body of the digit tourniquet may adhere to itself, securing it in position. A cover element, which is removably overlayable on the adhesive portion, may then protect the adhesive from premature adherence to any contacting surfaces. The cover element may be at least partially coated with a release agent such that the cover element is easily removable.

The adhesive portion may preferably be disposed between the engagement region and the release element, thus preventing the adhesive from interfering with the operation of the release element.

Preferably, the adhesive may include waterproof adhesive, such that the fastening is not affected by blood or other water-based fluids, and/or pressure-sensitive adhesive, such that it may be fastened with only the use of pressure.

In a preferred arrangement, the engagement region may include a flap, which is moveable to produce an aperture through the main body. During engagement, the flap folds back upon itself, forming a smooth surface on which the second portion may slide. A weakened portion may be provided at an interface between the flap and first portion, in order to encourage articulation of the flap. Preferably, this weakened portion may include a flap perforation.

Alternatively, the engagement region may include an aperture through the main body. The aperture may preferably be at least as wide as a lateral extent of the second portion. As such, the second portion may pass through the aperture without undue restriction.

Preferably, the release element may include a release perforation. Perforations are commonly used to weaken materials and provide a controlled and predictable path for the rupture to occur. Additionally or alternatively, at least one cut-out portion may be provided on the main body, for initiating rupture. The or each cut-out portion acts to concentrate stresses in the material, such that breakage may easily be triggered, when required.

Beneficially, the release element may include a tab for manually inducing rupture of the main body. A tab can be easily manipulated by a user such that a force can be applied.

Preferably, the inextensible or substantially inextensible material may include at least one of plasticised paper material, polyethylene, or polypropylene.

In a preferred arrangement, the quick-release digit tourniquet may further comprise a tension-limiting element for forming a rupture traversing the lateral extent of the main body when placed under a predetermined tension. This tension-limiting element may be incorporated within the release element.

The quick-release digit tourniquet preferably further includes a third portion releasably engaged with the first portion and spaced from the second portion, the third portion including an indicator for an instrument/device tally. This enables the tourniquet to be counted out, thereby preventing loss or misplacement. In this case, the third portion may be connected to the first portion via perforations. Additionally or alternatively, the first, second and third portions are preferably integrally formed as one-piece.

According to a second aspect of the invention there is provided a tourniquet strip comprising a plurality of quick-release digit tourniquets in accordance with the first aspect of the invention, the said quick-release digit tourniquets being detachably connected to one another.

Preferably, the plurality of quick-release digit tourniquets may be detachably connected in a side-by-side configuration. Alternatively they may be detachably connected in an end-to-end configuration.

According to a third aspect of the invention there is provided a method of releasing a quick-release digit tourniquet, comprising the steps of: a] providing a quick-release digit tourniquet having an elongate main body of a inextensible or substantially inextensible material having interengagable first and second portions; an engagement region located on the first portion of the main body through which the second portion may pass; and a release element for weakening the main body such that a transverse rupture of the main body is formed under application of a predetermined external force; b] fastening the quick-release digit tourniquet around a body part of a user; and c] applying an external force to the release element to manually initiate the rupture of the release element such that the quick-release digit tourniquet is no longer fastened around the said body part of the user.

Preferably, the release element may include a tab, the rupture of the release element being initiated by applying a force to the tab.

According to a fourth aspect of the invention there is provided a load-limiting tourniquet for preventing over-tightening comprising: an elongate main body of an inextensible or substantially inextensible material having interengagable first and second ends; and a tension-limiting element, laterally bisecting the main body and configured to rupture when placed under a predetermined tension threshold.

According to a fifth aspect of the invention there is provided a method of preventing over-tightening of a tourniquet, the method comprising the steps of: a] providing a quick-release tourniquet having an elongate main body of an inextensible or substantially inextensible material having interengagable first and second portions and a tension-limiting element for forming a transverse rupture of the main body when placed under a predetermined tension; b] fastening the load-limiting tourniquet around a body part of a user, the fastening process resulting in a tension being applied to the load-limiting tourniquet; and c] the tension-limiting element spontaneously rupturing when the tension is above the said predetermined tension.

### BRIEF DESCRIPTION OF THE DRAWINGS

The invention will now be more particularly described, by way of example only, with reference to the accompanying drawings, in which:

FIG. 1 shows a plan view of a first embodiment of a quick-release digit tourniquet, in accordance with the first aspect of the invention;

FIG. 2 shows an enlarged plan view of a first portion of a second embodiment of a quick-release digit tourniquet in accordance with the first aspect of the invention;

FIG. 3 shows a perspective view of a third embodiment of a quick-release digit tourniquet in accordance with the first aspect of the invention, the tourniquet being in an engaged configuration;

FIG. 4 depicts a portion of a tourniquet strip including a plurality of quick-release digit tourniquets as shown in FIG. 1; and

FIG. 5 is a fourth embodiment of a quick-release digit tourniquet in accordance with the first aspect of the invention, incorporating an integrated indicator or counter.

### DETAILED DESCRIPTION OF PREFERRED EMBODIMENTS

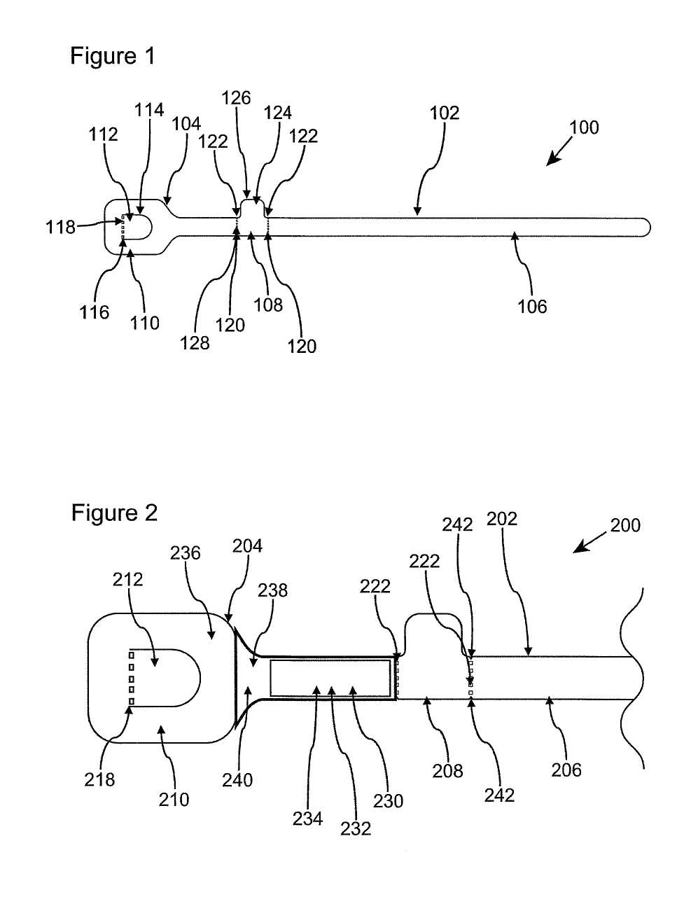

Referring firstly to FIG. 1 of the drawings, there is shown a first embodiment of a quick-release digit tourniquet 100 comprising an elongate main body 102, having first and second portions 104, 106, and a release element 108 interposed therebetween. The second portion 106 preferably has a uniform or substantially uniform lateral extent along at least a majority of its longitudinal extent, whilst the first portion includes an enlarged engagement region 110, which is preferably wider than the lateral extent of the second portion 106. The first portion 104 can thus be said to define a head, and the second portion 106 can be said to define a tail.

Typically, but not necessarily, the lateral extent of the second portion 106 may be 6 mm whilst the length of the main body 102 may be around 182 mm such that it may extend around the typical range of a human digital circumference whilst retaining sufficient length to tighten and fasten the tourniquet 100. Other lengths are also possible, depending on the size of the digit requiring treatment. For instance, the length could range from 100 mm to 225 mm, preferably, from 150 to 200 mm, and most preferably from 170 mm to 190 mm. Such a length should preferably be capable of use around the majority of human fingers and/or toes.

The main body 102 may preferably be formed of a single layer of an inextensible or substantially inextensible material, preferably being plasticized. Such materials may include polyethylene, polypropylene, and plasticised paper material. Most beneficially, the main body 102 may be a single-layer high density polyethylene film, which is preferably print receptive, satin finished, white and/or with a thickness of 200 microns. Such an arrangement is particularly beneficial for medical applications involving wet or damp conditions, and where a degree of lateral and longitudinal rigidity whilst retaining flexibility may be required. Such semi-rigidity enables the in use main body to spread its applied pressure without necessarily resulting in undue curvature particularly in the lateral extent causing increased point pressure on a user's skin.

The film is also preferably corona treated, and more preferably in-line corona treated, thereby enabling enhanced print characteristics, typically by utilising letterpress flexographic, screen and litho processes.

Advantageously, providing the main body 102 as a single layer of such materials ensures that delamination of multiple layers, which is known to occur as a consequence of bending or folding tourniquets for transit, is prevented. However, where the prevention of delamination is not a priority, the main body may also be formed of multiple layers of a single material, or a composite of different materials, resulting in different properties.

Within the engagement region 110 is preferably a flap 112, such as a buckle flap, openable to define an aperture 114, through which the second portion 106 may pass to engage with the first portion 104, forming a loop. The flap 112 is joined at an interface between the first portion 104 and the flap 112 by a weakened portion 116. The weakened portion 116, which in this embodiment is a flap perforation 118, acts to encourage the articulation of the flap 112 portion in a reliable and repeatable manner. The weakened portion 116 may be or may alternatively be a hinge to enable pivoting of the flap 112. The weakened flap 112 is beneficial in that it enables simplified removal to assist a scrub nurse or similar during an instrument count.

The engagement region 110 may preferably be sized such that the area surrounding the aperture 114 is a constant width, which is equal or substantially equal to that of the secondary portion 106. As such, there may be no concentration of stress around the aperture 114, which could result in premature and unwanted failure. The width of the main body 102, including the area around the engagement region 110 may vary from 2.5 to 10 mm, again dependent on the required strength and structural integrity of the tourniquet 100. However, the width should preferably be between 5 and 7 mm.

The release element 108 of the present embodiment comprises at least one and in this case preferably two weakened areas 120, shown herewith as release perforations 122, which are disposed either side of a lateral extension 124 of the main body 102, forming a tab 126. The tab 126 is sized such as to be easily operable by a user, preferably manually. In use, in order to detach the quick-release digit tourniquet 100, the user need only apply a force to the tab 126, which is transferred to the release element 108. This force causes the release element 108 to rupture, in this embodiment along the release perforations 122. As such, the main body 102 of the digit tourniquet 100 is laterally or transversely separated, and thus detached or detachable from a patient.

The force provided in order to cause rupture of or at the weakened areas 120 may be predetermined by the design of the weakened areas 120 themselves, or otherwise by the material properties. Other methods of predetermining the rupture force may be known by the skilled person. In any event, the rupture force is less at the weakened areas 120 than at the remainder of the longitudinal extent of the main body 102.

Advantageously, a tension-limiting element 128 may be included. In the present embodiment, the tension-limiting element 128 is incorporated with the release element 108 in the release perforations 122. The tension-limiting element 128 is designed to fracture, rupture, or break in response to the main body 102 of the digit tourniquet 100 being subjected to a tension above a predetermined threshold. As such, the tension-limiting element 128 can prevent the digit tourniquet 100 from exposing a patient to an unsuitable pressure, which could result in damage or further injury.

The tension-limiting element 128 could be designed to rupture at a specified safe pressure in order that the tourniquet 100 fails and is released from the patient. This can be advantageously applied through the number of perforations used. Alternatively, the tension-limiting agent could be constructed from an extensible material such that the tension is spontaneously released by extension of the tension-limiting element, thus keeping the applied tension below a prescribed limit.

A second embodiment of a quick-release digit tourniquet 200 is depicted in FIG. 2. Features which are similar or identical to those of the first embodiment are given similar or identical references, and further detailed description has been omitted, for brevity.

Furthermore, the second portion 206 of this embodiment is identical to that of the first embodiment, and thus this portion is omitted from the drawing.

The second embodiment includes first and second portions 204, 206 of the main body 202, being a head and tail, respectively, as in the first embodiment and again interposed by a release element 208. An engagement region 210, including a flap 212 and flap perforation or hinge 218, is also included. However, in this embodiment a fastener 230 is provided between the engagement region 210 and the release element 208.

The fastener 230 comprises an adhesive portion 232 having adhesive 234 disposed thereon. This adhesive portion 232 is on a face 236 of the main body 202 and is positioned as such in order that the second portion 206, when in an engaged condition with the first portion 204, may then be fastened to the adhesive portion 232, securing the engagement.

Also shown in FIG. 2 is a cover element 238, overlaying the adhesive portion 232. In the present embodiment, the cover element 238 is transparent, but it could also be translucent or opaque, if preferred. This cover element 238 is beneficial, before use, in that it may prevent the adhesive 234 prematurely adhering to any contacting surfaces. The cover element 238 is preferably large enough to overlay the entire adhesive portion 232, and more preferably may be larger than the adhesive portion 232 in order that an overlap 240 is provided which further protects the adhesive portion 232. The cover element 238 may be removed by a user, prior to or during application of the digit tourniquet 200 to a patient. As such, the digit tourniquet 200 may be readied for fastening.

Clearly shown in the enlarged view of FIG. 2, two cut-out portions 242 are formed adjacent to the release perforations 222. These cut-out portions 242 act to concentrate stress in the main body 202. As such, when a force is applied to the release element 208, the concentrated stress causes easier rupture of the release element 208 and thus separation of the main body 202. As such, a lower force is required to remove the digit tourniquet 200 from a patient.

The cut-out portions 242, which may be considered notches or slits, may be provided in opposing pairs, as depicted, or may instead be provided independently. Furthermore, the release perforations 222 may be omitted entirely, if required, as the cut-out portions 242 provide effective weakening of the main body 202, which may still allow rupture to be initiated.

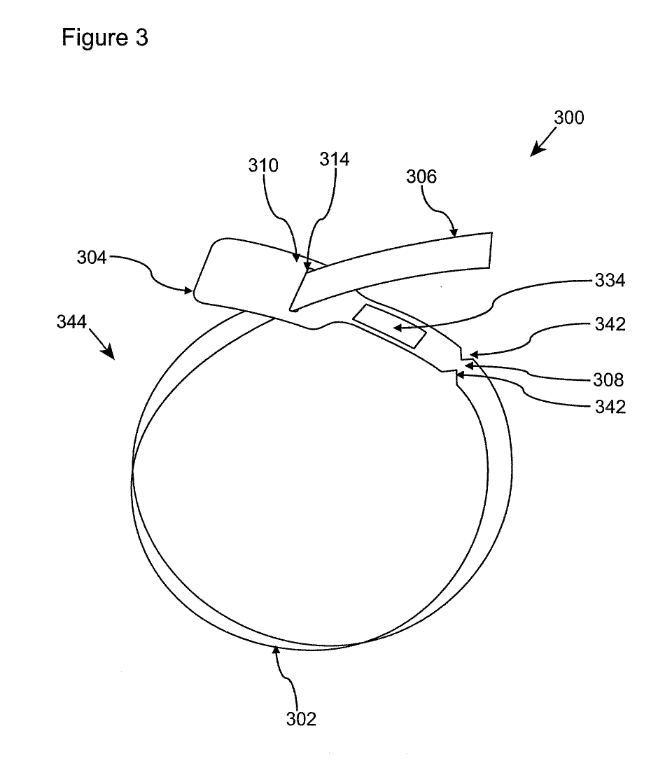

An engaged condition of a third embodiment of the quick-release digit tourniquet 300 is shown in FIG. 3. Like or similar features are given like or similar references, and thus further detailed description is again omitted, for brevity.

The second portion 306 of the digit tourniquet 300 is inserted through the aperture 314 of the engagement region 310 of the first portion 304, the main body 302 thus forming a loop 344. Though the present embodiment is not shown in a fastened condition, such a condition would be provided by bringing the second portion 306 into contact with the adhesive 334, once the desired length and/or tension of loop 344 has been achieved. As can be seen, the release element 308 of the third embodiment comprises two opposing cut-out portions 342. As in all the embodiments, it is preferred that the head is oversized or wider in a lateral extent than the tail. However, it is feasible that the head and tail may have a similar or identical lateral extent whereby the tail is curved about its longitudinal axis to be engaged with the head.

A tab is not provided, however, and therefore it is anticipated that the release element 308 will be triggered by rotating the first portion or head 304 relative to the second portion or tail 306 or vice versa. Whilst such a design may result in the release element 308 being slightly more difficult to activate, the manufacture of the simpler design of the third embodiment will be more cost-effective.

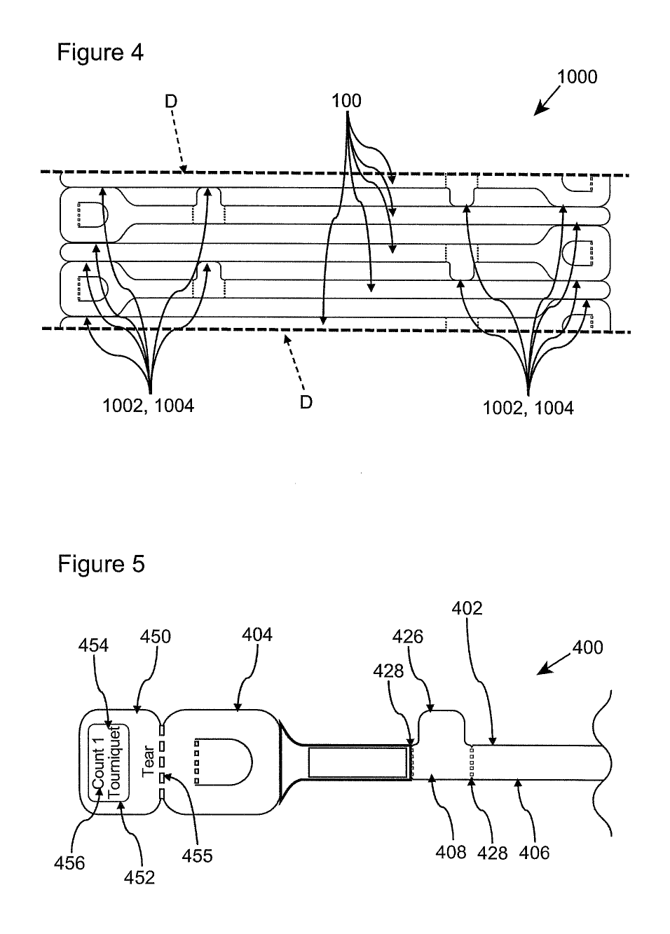

FIG. 4 shows a portion of an embodiment of a tourniquet strip 1000. A total of five digit tourniquets 100 are visible in the tourniquet strip 1000, although a greater or lesser number may be provided, depending on need or manufacturing capability. Dotted lines D, indicate that the strip may continue in either direction, and that what is shown is only a limited view. Each digit tourniquet 100 is releasably connected to two other digit tourniquets 100, in a longitudinal side-by-side arrangement. As such, either two or four joints 1002 are formed between each pair of adjacent digit tourniquets 100. The number of joints 1002 between adjacent tourniquets 100 is dependent on their individual orientations, and therefore a greater or lesser number of joints 1002 may be provided.

By providing the quick-release digit tourniquets 100 in such a manner, the manufacture can be made more efficient, with less wastage of material compared with producing the devices individually. Furthermore, the delivery and packaging can be optimised by, for instance, providing a tourniquet strip 1000 which has been formed on a roll, such that a single digit tourniquet 100 may be detached whenever needed. Alternatively, sheets of tourniquet strips 1000 may be manufactured, which may then be easily transported in small packages.

In the tourniquet strip 1000 of the present embodiment the joints 1002 may be formed as scores 1004 in the material, thus making the individual digit tourniquets 100 easily separable by a user. The joints 1002 could also be provided as perforations or other weakenings. Otherwise, it may also be possible to provide no weakening whatsoever, such that the digit tourniquets 100 must be separable by a knife, scissors, or other cutting device.

Though shown conjoined in a side-to-side arrangement, it may also be possible to provide the quick-release digit tourniquets in an end-to-end or other such arrangement. Preferably, each digit tourniquet should be separated from adjacent digit tourniquets by as little material as possible, so as to minimise wastage. Other arrangements may also be achievable, which will be apparent to the skilled person.

Whilst described herewith as separate elements, the release element and tension-limiting element may be one and the same element. Due to each of them working through forming a weakened area of the main body, they effectively work in the same way. However, it is advantageous for the tension-limiting element to be specifically designed to break at a set tension, whilst the release element should be preferably, though not necessarily exclusively, manually activated.

Referring now to FIG. 5, there is shown a fourth embodiment of a resiliently-flexible quick-release digit tourniquet 400 which, as in the previous embodiments, comprises an elongate main body 402 formed of an inextensible or substantially inextensible material, and has first and second, preferably head and tail, portions 404, 406, along with a release element 408 interposed therebetween. As above, features which are similar or identical to those of the previous embodiment are given similar or identical references, and further detailed description has been omitted. Furthermore, the second portion 406 is identical to that of the first embodiment, and thus the remainder of this portion is omitted from the drawing.

In an addition to these features, a third portion 450 in the form of a secondary head is provided at or adjacent to the first head portion 404, preferably being opposed to the second tail portion 406. The secondary head 450 is releasably engaged via a perforated connection 455 or other suitable release means.

An indicator 452 is provided on the secondary head 450. In this case, the indicator 452 comprises a display region 454 and one or more indicia 456 on the display region 454 indicating a count number of the digit tourniquet, whereby a scrub nurse or other medical practitioner can tally instruments and devices to be used.

Beneficially, the quick-release digit tourniquet 400 may be integrally formed as one-piece with the secondary head 450. As such, it is preferable that the quick-release digit tourniquet 400 and secondary head 450 are coplanar and formed as a single layer of, preferably plastics, material.

As above, it is preferable that the release element 408 includes a pull tab 426 for gripping by a user to release the in use digit tourniquet 400, and/or tension-limiting element 428 to enable rupturing beyond a given predetermined tension.

Although the third portion 450 is preferably a head similar in shape to the first portion, it may be a secondary tail or other suitable shape.

Adhesive used in the present invention, and particularly within the adhesive portion, may include particular properties to give specific advantages to the quick-release digit tourniquet. For example, use of a waterproof adhesive may be beneficial, as the adhesive will not be affected by any blood or other water-based fluid which may come into contact with the tourniquet. This can prevent or limit premature failure of the device. Additionally or alternatively, use of a pressure-sensitive will ensure that the main body may be fastened onto itself with only the application of pressure by a user, rather than requiring any setting agent or other such measure. Furthermore, a directional adhesive may give the tourniquet further beneficial characteristics. For instance, forces tangential to the main body, produced by the pressure of the digit pressing outwards may be absorbed strongly, whilst normal forces, such as those applied by a user to peel the two portions of the main body away from each other, may be only wealdy resisted by the adhesive. This can allow the quick-release tourniquet to be repositionable by a user, without resorting to breakage of the release element.

Although the present invention has been described throughout as a digit tourniquet due to the inherent problems associated with haemostasis in fingers and toes, the concept of providing a release element may be extended to tourniquets also suitable for use on limbs or other extremities. In such cases, the quick-release tourniquet will be necessarily larger or smaller, depending on the body part requiring treatment. Therefore, alterations may be required to the dimensions, thickness of the main body, material choice, or other characteristics, though the generally disclosed features may remain the same.

Whilst the preferred dimensions of a digit tourniquet in accordance with the invention have been herewith disclosed, other dimensions may also be used, the limits being only the size of the digit requiring treatment and the pressure that is required to be exerted.

It is therefore possible to provide a digit tourniquet having a release element rendering it swiftly and easily removable from a patient. The release element may be triggered by a manually-applied force from a user, such that the main body of the digit tourniquet is split into at least two parts.

It is also possible to provide a digit tourniquet which spontaneously ruptures when a tension is applied which is above a certain threshold. Furthermore, a tourniquet strip formed of a plurality of conjoined digit tourniquets may also be provided, such that they may be manufactured, transported, and used in an economical manner.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention herein described and defined.

What is claimed is:

1. A quick-release digit tourniquet for simplified removal from a digit, the digit tourniquet comprising:
an elongate main body of an inextensible or substantially inextensible material having interengagable first and second portions; and
a release element interposed between the first and second portions, which weakens the main body such that a transverse rupture of the main body is formed under application of a predetermined external force.

2. The quick-release digit tourniquet as claimed in claim 1, further comprising an engagement region, located on the first portion, through which the second portion may pass.

3. The quick-release digit tourniquet as claimed in claim 2, further comprising a fastener on the main body which secures the second portion once it has passed through the engagement region, resulting in a fastened condition.

4. The quick-release digit tourniquet as claimed in claim 2, wherein the fastener includes an adhesive portion, having an adhesive thereon, disposed on a face of the main body, a cover element removably overlayable on the adhesive portion, and wherein the cover element is at least partially coated with a release agent.

5. The quick-release digit tourniquet as claimed in claim 4, wherein the adhesive portion is disposed between the engagement region and the release element.

6. The quick-release digit tourniquet as claimed in any one of claims 4, wherein the adhesive is one of a waterproof adhesive and a pressure-sensitive adhesive.

7. The quick-release digit tourniquet as claimed in claim 1, wherein the engagement region includes a flap which is moveable to produce an aperture through the main body.

8. The quick-release digit tourniquet as claimed in claim 7, further comprising a weakened portion at an interface between the flap and the first portion, the weakened portion including a flap perforation.

9. The quick-release digit tourniquet as claimed in claim 1, wherein the engagement region includes an aperture through the main body which is at least as wide as a lateral extent of the second portion.

10. The quick-release digit tourniquet as claimed in claim 1, wherein the release element includes at least one of release perforations and at least one cut-out portion on the main body, which initiate rupture.

11. The quick-release digit tourniquet as claimed in claim 1, wherein the release element includes a tab which manually induces rupture of the main body.

12. The quick-release digit tourniquet as claimed in claim 1, wherein the inextensible or substantially inextensible material includes plasticised paper material, polyethylene, or polypropylene.

13. The quick-release digit tourniquet as claimed in claim 1, further comprising a tension-limiting element which forms a rupture traversing the lateral extent of the main body when placed under a predetermined tension.

14. The quick-release digit tourniquet as claimed in claim 13, wherein the tension-limiting element is incorporated within the release element.

15. The quick-release digit tourniquet as claimed in claim 1, further comprising a third portion releasably engaged with the first portion and spaced from the second portion, the third portion including an indicator for an instrument/device tally, wherein the third portion is connected to the first portion via perforations.

16. A load-limiting tourniquet for preventing over-tightening, comprising:
an elongate main body of an inextensible or substantially inextensible material having interengagable first and second ends, the inextensible or substantially inextensible material being at least one of plasticised paper material, polyethylene, and polypropylene;
a tension-limiting element including at least one of tension-limiting perforations and one or more cut-out portions, a tab for manually inducing rupture of the tension-limiting element, and which laterally bisects the main body and configured to rupture when placed under a predetermined tension threshold;
an engagement portion, located at or adjacent to the first end, through which the second end may pass; and
an adhesive fastener with a cover element on the main body disposed between the engagement region and the tension-limiting element for securing the second end once it has passed through the engagement portion, resulting in a fastened condition.

17. A method of preventing over-tightening of a tourniquet, the method comprising the steps of:
providing a quick-release tourniquet having an elongate main body of an inextensible or substantially inextensible material having interengagable first and second portions and a tension-limiting element which forms a transverse rupture of the main body when placed under a predetermined tension;
fastening the load-limiting tourniquet around a body part of a user, the fastening process resulting in a tension being applied to the load-limiting tourniquet; the tension-limiting element spontaneously rupturing when the tension is above the said predetermined tension.
